(19) United States
(12) Patent Application Publication
(10) Pub. No.: US 2011/0158950 A1
(43) Pub. Date: Jun. 30, 2011

(54) USE OF BLOOD GROUP STATUS I

(75) Inventors: Pirjo WACKLIN, Helsinki (FI); Jaana MÄTTÖ, Helsinki (FI); Harri MÄKIVUOKKO, Kirkkonummi (FI)

(73) Assignee: SUOMEN PUNAINEN RISTI VERIPALVELU, Helsinki (FI)

(21) Appl. No.: 12/843,404

(22) Filed: Jul. 26, 2010

(30) Foreign Application Priority Data
Dec. 28, 2009 (FI) 20096400

(51) Int. Cl. A61K 35/74; A61P 1/00; C12Q 1/02

(52) U.S. Cl. 424/93.4; 435/29

(57) ABSTRACT

Provided is a probiotic composition which is tailored based on the spectrum of bifidobacteria found in the intestine of at least one individual with non-secretor blood group phenotype. Further provided is a method of tailoring a probiotic composition based on the bifidobacteria found from the intestine of at least one non-secretor individual.

### FIELD OF THE INVENTION

The present invention relates to a probiotic composition which is tailored based on the spectrum of bifidobacteria found in the intestine of at least one individual with non-secretor blood group phenotype. The present invention further relates to a method of tailoring a probiotic composition based on the bifidobacteria found from the intestine of at least one non-secretor individual. The present invention also relates to use of the secretor status of an individual as a criterion for bifidobacteria-enriched probiotic supplementation. The present invention relates also to method of assessing the need of an individual for bifidobacteria-enriched probiotic supplementation by determining the secretory status of the individual. Also, the invention relates to a method of treating and/or preventing disorders related to unbalanced mucosal microbiota in an individual.

### BACKGROUND OF THE INVENTION

Bifidobacteria comprise the predominant intestinal microbiota in infants and they are adundant also in the adult population comprising up to 10% of the normal intestinal microbiota, although their numbers start to decline in the elderly. An individual is typically colonised with 1-4 bifidobacterial species (Mättö et al. J Appl Microbiol 2004, 98, 459-470). In addition to the individual variation, composition of bifidobacterial species varies between different age groups. B. longum biovar infantis, B. breve and B. bifidum are the most prevalent species in infants and B. longum biovar longum, B. adolescentis, B. bifidum and B. catenulatum in adults. Variation in the number of bifidobacteria (Mueller at al. Appl Environ Microbiol 2006, 72, 1027-1033) and composition of species (Mättö et al. 2004) between geographic regions has also been reported. Bifidobacteria are generally considered as health promoting bacteria and an increase in bifidobacterial numbers in the intestine is typically used as an end-point in intervention studies with intestinal health-targeted products such as probiotics and prebiotics.

Bifidobacterium spp. strains are used as probiotics. However, due to technological challenges related to stability of the genus, fairly few distinct species and strains, mainly B. animalis subps. lactis, are available on the current market. Bifidobacteria or bifidobacteria-containing strain mixtures have shown promising results e.g. in alleviation of the symptoms of irritable bowel syndrome (Brenner & Chey, Rev Gastroenterol Disord. 2009 Winter; 9(1):7-15), diarrhoea (Chouraqui et al. J Pediatr Gastroenterol Nutr. 2004 March; 38(3):242-3), atopic eczema (Yoo et al. Proc Am Thorac Soc (2007) 4, 277-282) and common cold (de Vrese et al. Clin Nutr. 2005 August; 24(4):479-80). Another challenge in addition to the above-mentioned stability problems, is the fact that a proportion of the study subjects usually have not responded to test probiotics or prebiotics (Fuccio et al., J Clin Gastroenterol 2009, 43, 506-513; Fujimori et al., J. Gastroenterol Hepatol 2007, 22, 1199-1204). These individual are often said to be ‘non-responders’. No reason behind the non-responsiviness is known.

The primary site of colonization of bifidobacteria is the colon, but they are also present in the oral cavity and have been isolated from human milk (Martin et al. Appl Environ Microbiol. 2009, 75(4):965-9). The major energy sources of bifidobacteria are non-digestible dietary carbohydrates and endogenous mucus. They are capable of degrading various oligosaccharides including human milk oligosaccharides and complex carbohydrates present in mucus as substrates. Several bifidobacteria have been shown to adhere to intestinal mucus (He et al. Microbiol Immunol 2001, 45, 259-262). Adhesion of Bifidobacterium bifidum to mucus has been shown to increase by supplementation of fucose (Guglielmetti et al. Curr Microbiol. 2009 August; 59(2):167-72). The vast variety and spectrum of microbial strains and species in the gut of mammals, including man and the findings demonstrating that the composition of microbial species in the gut will not directly predict their functional outcome, have indicated that predicting the functionality of single probiotic or normal flora species is difficult (Tap et al, Environm Microbiol 2009, 11, 2574-2584). The complexity of the ecosystem is simply too vast. The role of host genetic factors in determining the composition of normal gut microbiota is also poorly understood.

Binding to blood group antigens has been reported for certain single pathogenic species of bacteria and viruses. In particular, Helicobacter pylori binds to the Lewis b (Le^{b}) antigen in stomach (Boren et al. Science 1993, 262, 1892-1895) and Norovirus binds to ABH ja Le^{b} antigens (Huang et al. J Virol. 2005 June; 79(11):6714-22). Streptococcus pneumoniae has ability to bind A and B blood group antigens and utilise the glycans (Higgins et al. J Mol Biol. 2009 May 1; 388(2):299-309).

The blood group antigens are not present in the mucus of all individuals. These individuals, said to have ‘non-secretor’ blood group, do not have the functional FUT2 gene needed in the synthesis of secreted blood group antigens (Henry et al. Vox Sang 1995; 69: 166-182) and thus they do not secrete ABH antigens in secretions and on mucosa. Those with blood group ‘secretor’ have the antigens on mucosa. In most populations, the frequency of non-secretor individuals is substantially lower than that of secretor status, about 15-26% of Scandinavians are classified as non-secretors (Eriksson et al. Ann Hum Biol. 1986 May-June; 13(3):273-85). The secretor/non-secretor status can be regarded as a normal blood group system and the phenotype can be determined using standard blood banking protocols (Henry et al. 1995). The genotype, that is, the major mutation in the FUT2 gene causing the non-secretor (NSS) phenotype in the European populations (Silva et al. Glycoconj 2010; 27:61-8) has been identified. Non-secretor phenotype has been demonstrated to be genetically associated for example, with an increased risk for Crohn's disease (McGovern et al. Hum Molec Genet 2010 Advance Access Published Jun. 22, 2010), with high vitamin B12 levels in the blood (Tanaka et al Am J Hum Genet 2009; 84:477-482), with resistance to Norovirus infection (Thorven et al J Virol 2005; 79: 15351-15355), with susceptibility to HI virus infection (Ali et al 2000, J Infect Dis 181: 737-739), with experimental vaginal candidiasis (Hurd and Domino Infection Immunit 2004; 72: 4279-4281), with an increased risk for asthma (Ronchetti et al. Eur Respir J 2001; 17: 1236-1238), with urinary tract infections (Sheinfeld et al N Engl J Med 1989; 320: 773-777), and with an animal hemorrhagic disease virus (Guillon et al. Glycobiology 2009; 19: 21-28).

### BRIEF DESCRIPTION OF THE INVENTION

An object of the present invention is a microbial and/or probiotic composition which is tailored based on the spectrum of bifidobacteria found in the intestine of at least one individual with non-secretor blood group phenotype. Another object of the present invention is a method of tailoring a probiotic composition based on the bifidobacteria found from the intestine of at least one non-secretor individual. A further object of the invention is use of secretor blood group status of an individual in assessing the need for bifidobacteria-enriched probiotic supplementation, i.e., as a criterion for bifidobacteria-enriched probiotic supplementation. The present invention relates also to method of assessing the need of an individual for bifidobacteria-enriched probiotic supplementation by determining the secretory status of the individual. Also, an object of the invention is the use of prebiotics, molecular compounds or additional supportive bacteria strains, to increase the number of, and/or to augment the growth and/or functionality of bifidobacteria in the intestine.

A further object of the present invention is a use of the secretor blood group status of an individual in estimating a dose of bifidobacteria supplementation needed for a desired effect. Another further object of the present invention is to provide a method of identifying an individual at risk for suffering from a gastrointestinal disorder by determining the secretory status of said individual.

In addition, the invention relates to methods for treating and/or preventing disorders related to unbalanced mucosal microbiota and/or having FUT2 gene as a susceptible factor by administering to an individual an effective amount of the microbial composition of the present invention. Further, the invention relates to a method for treating and/or preventing inflammatory bowel disease and/or urogenital infections and/or low levels of vitamin B12 in an individual by administering to the individual an effective amount of the microbial composition of the present invention.

The invention is based on the observation that the individuals with non-secretor blood group phenotype have a reduced amount and/or a reduced diversity of bifidobacteria in their intestinal bacterial population as compared to those with the secretor phenotype. This observation can be used as a basis for targeted modulation of the bifidobacterial intestinal population in an individual, especially in a non-secretor individual. Accordingly, the current invention provides a novel and effective means for optimizing the bacterial, especially bifidobacterial content of a probiotic composition.

The objects of the invention are achieved by the compositions, methods and uses set forth in the independent claims. Preferred embodiments of the invention are described in the dependent claims.

Other objects, details and advantages of the present invention will become apparent from the following drawings, detailed description and examples.

### BRIEF DESCRIPTION OF THE DRAWINGS

FIG. 1 shows a DGGE gel image of bifidobacterial diversity on faecal samples of 7 non-secretor and 7 secretor individuals. M=marker. Each lane represents a single sample.

FIG. 2 illustrates the three-dimensional PCA plot based on the DGGE analysis of the bifidobacterial profiles.

FIG. 3 illustrates PCA biplot of bifidobacterial DGGE profiles showing the DGGE band positions, which most significantly contributed to the first and the second principal components explaining together 56.3% of the variance. Insert figure indicates the band positions, which contributed the principal component most. Non-secretor samples are indicated with dot, non-secretor with star and samples of unknown secretor status with square.

FIG. 4 illustrates the Shannon diversity Index based on bifidobacterial DGGE profiles test between secretor and non-secretor individuals. P-value for t-test between non-secretor and secretor individuals is shown.

FIG. 5 illustrates the identity of the band positions of Bifidobacteria-DGGE gels based on Blast search of the sequences. The excised and sequenced bands are marked with numbers. The bold letters show band positions, which were either absent or detected rarely in non-secretors. The identity of band positions is shown in the side of the gels with arrows and the colours of the numbers indicate the bands belonging to the same band position and having identical sequences: band position 26.6% (B. adolescentis) contains sequenced bands 15, 24, 27 and 29; band position 29.7% (B. bifidum) contains sequenced bands 6, 16, 20 and 32; band position 53.5% (B. lognum) contains sequenced bands 1, 3, 7, 9, 12, 21 and 33; band position 55.0% (Bifidobacterium sp.) contains sequenced bands 4 and 18; band position 62.2% (uncultured bifidobacterium) contains sequenced bands 1, 5, 13, 19, 25, 31 and 37; band position 63.7% (B. catenulatum/pseudocatenulatum) contains sequenced bands 22 and 34. Identity of the band positions, which were based on single sequence were following (in black): 8=Bifidobacterium sp. (B. catenulatum), 11=B. adolescentis, 17=uncultured bifidobacterium (B. ruminantium), 30=uncultured bifidobacterium (B. adolescentis), 36=uncultured bifidobacterium (B. ruminantium). The strain name in the parentheses indicates the closest cultivated relative of the sequence, if available.

FIG. 6 shows an image of the normalised DGGE profiles for non-secretor individuals, secretor individuals and individuals with unknown secretor status. Numbers in grey boxes and vertical lines indicate the band positions and star symbol on vertical line indicates that band was binned to the band positions.

### DETAILED DESCRIPTION OF THE INVENTION

As bifidobacteria comprise the predominant intestinal microbiota in infants and are adundant also in the adult population, they are considered as essential for maintaining and/or promoting health of an individual. High bifidobacterium diversity in the gut is beneficial for the health of an individual, because bifidobacteria can, for example, prevent adhesion of adverse microbes on gut epithelium and prevent their colonisation in the intestine. They may also modulate the immune response of the host.

The present invention is based on the finding that the individuals with non-secretor blood group have a reduced amount of bifidobacteria in their intestinal bacterial population. Further, the present invention is based on the finding that the non-secretor individuals have a reduced diversity of bifidobacteria genus in their intestinal bacterial population. These findings can be used as a basis for targeted modulation of the bifidobacterial population in the non-secretor individuals and as a criterion for bifidobacteria enriched probiotic supplementation.

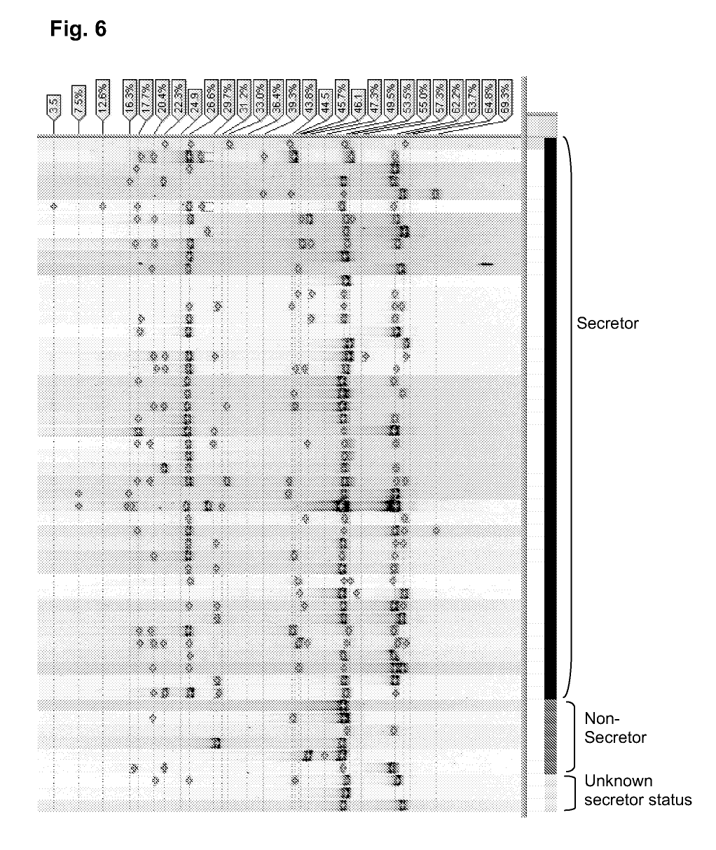

Bifidobacterium genotypes that were found to be present at least in one non-secretor individual are listed below in Table 1. The band positions are presented in detail in FIG. 6.

TABLE 1

| Band | |
|---|---|
| position | Genotype name |
| 16.3% | Bifidobacterium genotype 4 |
| 20.4% | Bifidobacterium genotype 6 |
| 22.3% | Bifidobacterium genotype 7 |
| 29.7% | Bifidobacterium bifidum |
| 43.8% | Bifidobacterium genotype 12 |
| 47.3% | Bifidobacterium genotype 16 |
| 49.5% | Bifidobacterium genotype 17 |
| 55.0% | Bifidobacterium genotype 18 |
| 62.2% | Bifidobacterium genotype 20 |
| 53.5% | B. lognum |

Bifidobacterium genotypes that were found to be present in secretor individuals and absent in non-secretor individuals are listed in Table 2. The band positions are presented in detail in FIG. 6.

TABLE 2

| Band | |
|---|---|
| position | Genotype name |
| 7.5% | Bifidobacterium genotype 1 |
| 3.5% | Bifidobacterium genotype 2 |
| 12.6% | Bifidobacterium genotype 3 |
| 17.7% | Bifidobacterium genotype 5 |
| 24.9% | Bifidobacterium genotype 8 |
| 26.6% | Bifidobacterium adolescentis |
| 31.2% | Bifidobacterium genotype 9 |
| 33.0% | Bifidobacterium genotype 10 |
| 39.3% | Bifidobacterium genotype 11 |
| 44.5% | Bifidobacterium genotype 13 |
| 45.7% | Bifidobacteriumgenotype 14 |
| 46.1% | Bifidobacterium genotype 15 |
| 57.3% | Bifidobacterium genotype 19 |
| 63.7% | B. catenulatum/pseudocatenulatum |
| 69.3% | Bifidobacterium genotype 21 |

The term ‘probiotic’ here refers to any bacterial species, strain or their combinations, with health supportive effects, not limited to currently accepted strains or to intestinal effects. The term ‘prebiotic’ here refers to any compound, nutrient, or additional microbe applied as a single additive or as a mixture, together with probiotics or without probiotics, in order to augment a desired probiotic health effect or to stimulate the growth and activity of those bacteria in the digestive system which are assumed to be beneficial to the health of the body.

The present invention relates to a microbial and/or probiotic composition which is tailored based on the spectrum of bifidobacteria found in the intestine of at least one non-secretor individual. Particularly, the present invention relates to a probiotic composition tailored based on the bifidobacterial composition of the intestine of at least one individual with non-secretor blood group phenotype.

In one embodiment, the microbial or probiotic composition comprises at least one of the strains listed in Table 1. In another embodiment, the probiotic composition comprises two or more of the strains listed in Table 1.

The present invention relates also to a method of tailoring a probiotic composition based on the bifidobacteria found in the intestine of at least one individual with non-secretor blood group phenotype.

The probiotic composition of the present invention and the probiotic supplement comprising the composition are particularly suitable and effective, but not limited to in use, for the non-secretor individuals for the enhancement of the diversity and numbers of intestinal bifidobacteria. The supplement is based on the rationale that those species of bifidobacteria that can be detected in non-secretors, can also attach themselves to and grow on the gut. Non-secretors have been reported to be more vulnerable for infections (Blackwell, C. C. 1989. The role of ABO blood groups and secretor status in host defences. FEMS Microbiology Immunology 47, 341-350). A balanced and diverse population of beneficial Bifidobacteria is, therefore, particularly important for non-secretors.

In one embodiment of the invention, the secretor/non-secretor status can be used to augment the stabilisation of mucosal microbial, especially Bifidobacterium composition of an individual after disorders or treatments known to disturb the balance of mucosal microbiota. Examples of these comprise treatments with strong antibiotics, irradiation or cytotoxic therapies related to cancer treatments or bone marrow transplantation and/or gastroenterological infections by e.g. Noro-virus or Helicobacter. The present invention is further targeted to treatment of diseases or traits, having the FUT2 gene (i.e. the secretor blood group status) as a genetic susceptibility factor. These comprise, just to give examples, low levels of vitamin B12 in the blood, various clinical forms of inflammatory bowel disease, urinary tract infections, vaginal candidiasis, Noro- and HI-virus infections, and infections by hemorrhagic viruses. It is likely that a higher number of diseases will be identified in the future by screening the FUT2 locus. Probiotic treatments typically are used to direct or change the microbiological balance in the gut toward more healthy one, or toward the microbial, especially bifidobacterial, spectrum “typical to individuals” with the non-susceptible FUT2 genotype. The present invention is particularly related to treatments directed to individuals with the non-secretor status. Individuals with the non-secretor phenotype typically require higher dosages and/or preparations with more diverse microbial strains than secretors. Thus, the present invention relates also to use of the secretor/non-secretor status of an individual to augment the stabilisation of mucosal Bifidobacterium composition in disorders related to, or after treatments leading to unbalance of mucosal microbiota. The present invention also relates to a method for treating and/or preventing disorders or diseases related to unbalanced mucosal microbiota in an individual by administering to the individual an effective amount of the microbial composition of the present invention. The present invention further relates to a method for treating and/or preventing disorders or diseases having FUT2 gene as a susceptible factor in an individual by administering to the individual an effective amount of the microbial composition of the present invention. In addition, the present invention relates to a method for treating and/or preventing inflammatory bowel disease, urogenital infections and/or low levels of vitamin B12 in an individual by administering to the individual an effective amount of the microbial composition of the present invention.

In one embodiment of the invention, the probiotic composition or a supplement comprising the composition is tailored for infants of the non-secretor type. In another embodiment, the probiotic composition or a supplement comprising the composition is tailored for infants regardless of their secretor phenotype, whose breast-feeding mother is of the non-secretor blood group type. The probiotic composition or a supplement comprising the composition can be used to enhance the development of a balanced intestinal microbiota composition. Babies of non-secretor mothers are more vulnerable to infections, because the milk of the mother does not contain fucosylated glycans, which act as binding locations for pathogens. The diet of babies of non-secretor mothers could be supplemented with fucosylated glycans as prebiotics, together with or without the bifidobacterium composition of the invention. A typical prebiotic ingredient is an oligo/polysaccharide which is non-digestible in the upper parts of the oro-gastrointestinal tract. These oligosaccharides include, but are not limited to, fructo-oligosaccharides or inulin, galacto-oligosaccharides, soy oligosaccharides, resistant starch, and polydextrose. An example shown to be particularly suitable for Bifidobacteria is lacto-N-biose I (Kiyohara et al., Biosci Biotechnol BioChem 2009; 73: 1175-1179). Prebiotics typically are produced by processing from natural sources e.g. from chicory root or milk, alternatively, they may be chemically synthesized. The daily dose needed for a prebiotic effect is typically several grams per day.

Additionally, in one embodiment, the invention is related to probiotic composition targeted to elderly individuals for supporting the maintenance of bifidobacteria diversity and abundance.

The probiotic compositions and supplements so designed may have beneficial effects on the health and/or well-being of a human and may be in the form of, for example, a food product, capsule, tablet or powder. The composition can be formulated into a product of dairy or beverage industry, a functional food product or a nutritional supplement as well as a capsule, emulsion, or powder.

A typical probiotic ingredient is freeze-dried powder containing typically 10¹⁰-10¹² viable probiotic bacterial cells per gram. In addition it normally contains freeze drying carriers such as skim milk, short sugars (oligosaccharides such as sucrose or trehalose). Alternatively, the culture preparation can be encapsulated by using e.g. alginate, starch, xanthan as a carrier. A typical probiotic supplement or capsule preparation contains approximately 10⁹-10¹¹ viable probiotic bacterial cells per capsule as a single strain or multi-strain combination.

A typical probiotic food product, which can be among others fermented milk product, fermented milk-based product or juice, contains approximately 10⁹-10¹¹ viable probiotic bacterial cells per daily dose. Probiotics are incorporated in the product as a probiotic ingredient (frozen pellets or freeze dried powder) or they are cultured in the product, such as yogurt, curd and/or sour milk, during fermentation.

Bifidobacteria containing composition or supplement contains optionally also at least one prebiotic optimised for the growth stimulation of the selected Bifidobacterium strain or strains. The addition of a prebiotic to the composition of the present invention is to further augment the efficacy of the probiotic composition by helping the survival of those Bifidobacterium spieces added into the composition but not commonly found in an individual.

The present invention provides also means for tailoring and/or optimising or potentiating an existing probiotic and/or synbiotic product with at least one bifidobacterial strain selected according to the present invention to improve the responsiveness and/or effect of the product in non-secretors.

The present invention also relates to a use of the secretory status of an individual in assessing the need for bifidobacteria-enriched probiotic supplementation. The present invention also relates to a method of assessing the need of an individual for bifidobacteria-enriched probiotic supplementation by determining the secretory status of the individual.

The present invention further relates to a use of the secretory status of an individual in estimating a dose of bifidobacteria supplementation needed for a desired effect. Typically individuals of non-secretor phenotype should need higher doses of probiotics than those with the secretor phenotype.

The present invention also relates to a method of identifying an individual at risk for suffering from a gastrointestinal disorder by determining the secretory status of said individual. The status can be determined, for example, from a sample of saliva, using standard blood grouping methods or from the genomic DNA of an individual by determining adequate mutations in the FUT2 gene (Silva et al. Glycoconjugate Journal 2009, DOI 10.1007/s10719-009-9255-8).

Stabilization of the intestinal bacterial population, especially bifidobacterial population, has been observed to be delayed after severe microbiota disturbances (Mättö et al. 2008). Thus, the present invention provides a use of the secretor status and bifidobacterial species diversity of an individual in following the microbiota stabilisation after such drastic disturbances.

The results of the present invention indicated that non-secretors had lower bifidobacterial diversity in the intestine than secretor individuals. Among strains of Bifidobacterium there were strains, yet to be identified at the genotype level, that were more common in the intestine of non-secretors. The non-secretors lacked or carried very low or undetectable numbers of several Bifidobacterium strains (e.g. B. adolescentis and B. catenulatum/pseudocatenulatum), which were common in secretors. Moreover, B. bifidum and certain bifidobacteria with still unidentified genotypes, were present more rarely in non-secretors than in secretors. Of the most frequently detected bifidobacterial strains, only B. longum was equally common in both secretors and non-secretors. Accordingly, some bifidobacteria are present in GI tract of almost all humans, but non-secretors miss some or many of the bifidobacterial strains i.e. all human share some bifidobacterial species, but non-secretors miss many bifidobacterial species present commonly in secretors. Based on these findings, the probiotic composition and/or supplement of the present invention contain in particular those bifidobacterial species abundant in individuals with non-secretor phenotype.

The invention will be described in more detail by means of the following examples. The examples are not to be construed to limit the claims in any manner whatsoever.

### EXAMPLES

### Materials and Methods

The materials and methods described herein are common to examples 1 to 7.

59 healthy adult volunteers (52 females and 7 males) we recruited to the study. Both faecal and blood samples were collected from 59 volunteers. The age of the volunteers ranged from 31 to 61 and was in average 45 years.

Faecal samples were frozen within 5 hours from defecation. DNA from 0.3 g of faecal material was extracted by using the FASTDNA® SPIN KIT FOR SOIL (Qbiogene). Partial bifidobacterial 16S rRNA gene was amplified by PCR with bifidobacterial specific primers Bif164F and Bif662R+GC (Satokari et al., Appl Environm Microbiol 2001, 67, 504-513). The specificity of the primers was tested with Bifidobacterium strains (B. adolescentis E-981074, B. bifidum E-97795, B. lactis E-97847, B. longum E-96666, B. angulatum DSM 20098 and Bifidobacterium catenulatum DSM 16992), which are the most common Bifidobacterium species inhabiting human gut, as well as 43 other bacterial strains having representatives of common human cut bacteria. Amplified PCR fragments were separated in 8% DGGE gel with denaturing gradient from 45% to 60%. DGGE gels were run at 70 V for 960 mins. DGGE gels were stained with SYRBSafe for 30 mins and documented with Safelmager Bluelight table (Invitrogen) and AplhaImager HP (Kodak) imaging system.

Digitalised DGGE gel images were imported to the Bionumerics-program version 5.0 (Applied Maths) for normalisation and band detection. Bands were normalised with marker samples constructed from bifidobacterial strains. Band search and bandmatching was performed as implemented in Bionumerics. Bands and bandmatching were manually checked and corrected.

The bands were excised from bifidobacteria-DGGE gels. DNA from bands was eluted by incubating bands in 50 μl sterile H₂O at +4° C. overnight. The correct position and purity of only each of the excised bands were tested by amplifying DNA in bands and running the amplified fragments along the original samples in DGGE. Bands, which only produced single bands and were in the correct position in the gels, were sequenced in Eurofins MWG (Germany). The sequences were trimmed, manually checked and corrected for ambiguous bases and aligned by ClustalW. The closest relatives of the sequences were searched using Blast and NCBI nr database. Distance matrix of the aligned sequences was used to compare the similarity of the sequences.

### Example 1

Secretor status was determined from the blood samples using the standard in-house blood grouping protocols of Finnish Red Cross Blood Service. Secretor status was determined from 59 individual and 48 were secretors and seven were non-secretors. For 4 samples, secretor status was could not be determined.

### Example 2

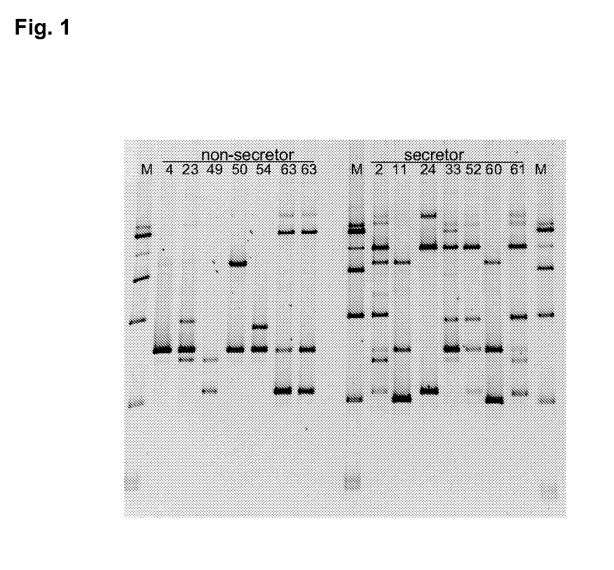

DGGE analysis targeted for the faecal bifidobacterial population was performed as described above in the material and methods. DGGE gel images showed fewer numbers of bands in the samples obtained from the non-secretor individuals than in the samples from secretor individuals, indicating that fewer bifidobacterial genotypes were present in non-secretor than in secretor individuals. In average, non-secretors had 2.5 (maximum 4) bands and secretors 5.2 bands (maximum 11 bands) in bifidobacterial DGGE profiles. In five samples bifidobacteria were not detected (one non-secretor sample and 4 secretor samples). The Bifidobacterial profiles of all non-secretor individuals and selected bifidobacterial profiles of the secretor individuals are presented in FIG. 1.

### Example 3

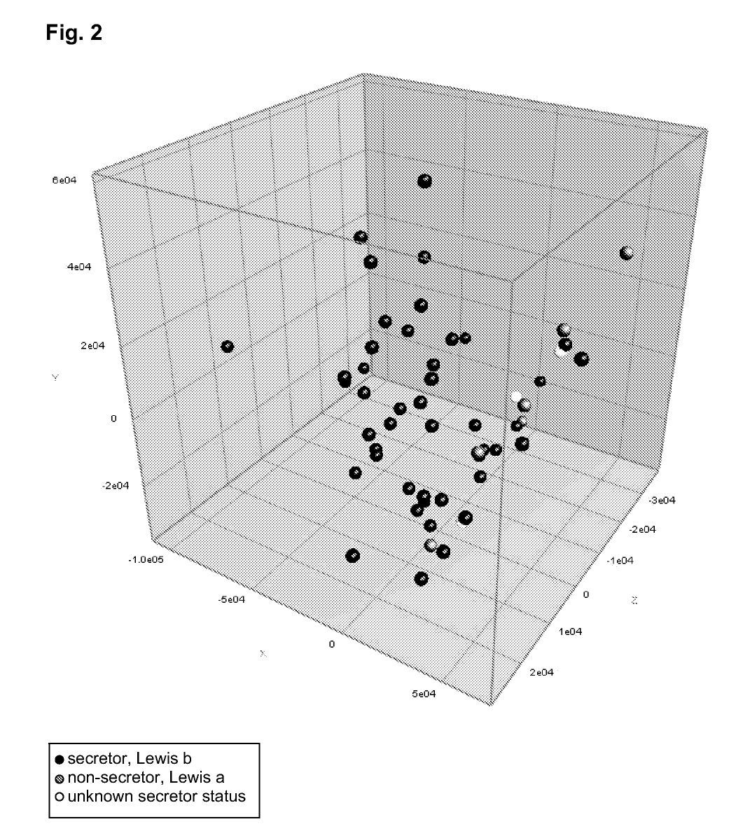

DGGE analysis targeted for the faecal bifidobacterial population was performed as described above. Principal component analysis (PCA) was performed as implemented in the Bionumerics software package. PCA based on intensities of bands detected by DGGE, was used to ordinate samples and to find out the bands which predominantly contributed to the principal components. Images of DGGE gels were analysed using the Bionumerics to allow statistical analysis between samples. PCA based on intensities of bands in DGGE gels showed grouping of the samples obtained from the non-secretors. The first and second principal component explained of the 56.3% of the total variance. The results are presented in FIG. 2.

### Example 4

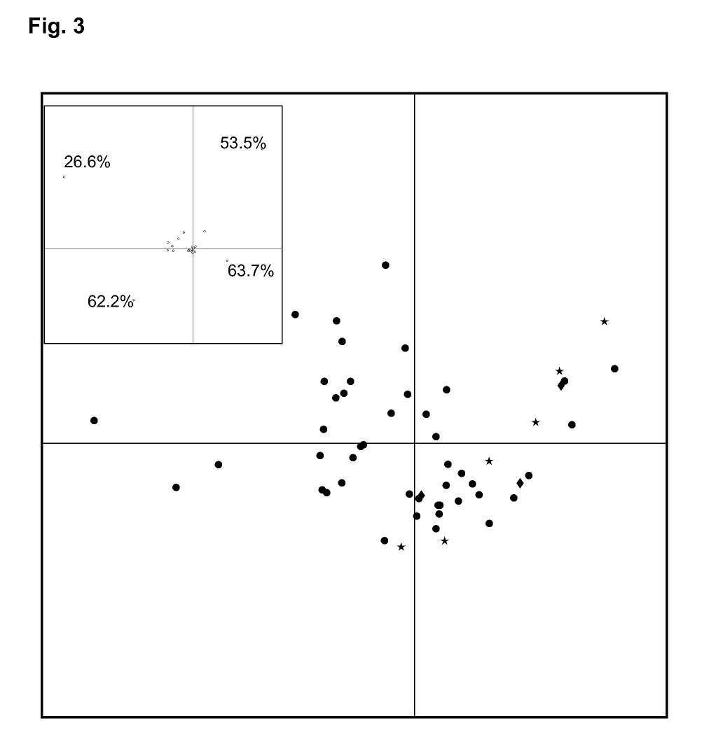

DGGE analysis targeted to the faecal bifidobacterial population was performed as described above. PCA based on intensities of bands detected by bifidobacterial DGGE was used to ordinate samples and to find out the bands which most contributed to the principal components. In the PCA biplot, the first and second principal component contributed 56.3% of the total variance. The bands in positions 26.6%, 53.3%, 62.2% and 63.7% contributed most clearly to the components. These bands were the most commonly detected bands in the samples (Table 3). The PCA biplot based on bifidobacterial DGGE profiles is presented in FIG. 3.

### Example 5

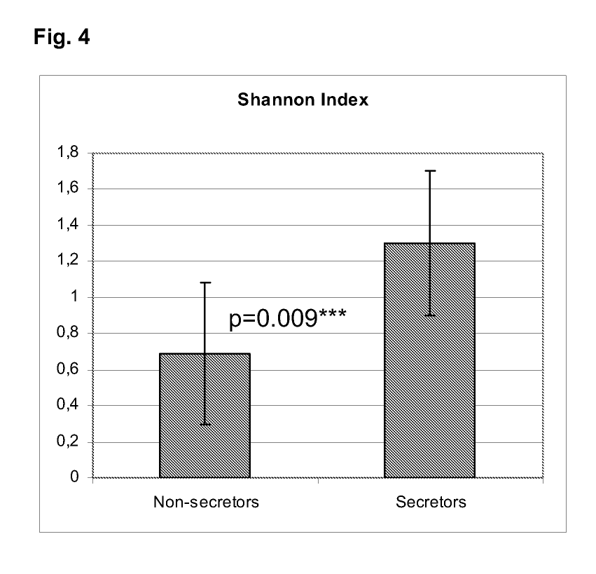

DGGE analysis targeted for the faecal bifidobacterial population was performed as described above. The Shannon diversity index based on band intensities was used to summarise the diversity of bifidobacteria in the samples. The index calculations and t-tests were done. The Shannon index, which describes diversity based on abundance and eveness of species, showed that bifidobacterial diversity was statistically significantly reduced in non-secretor individuals in comparison to secretor individuals (p=0.009). Thus, non-secretor individuals have lower bifidobacterial diversity than secretor individuals. The results are presented in FIG. 4.

### Example 6

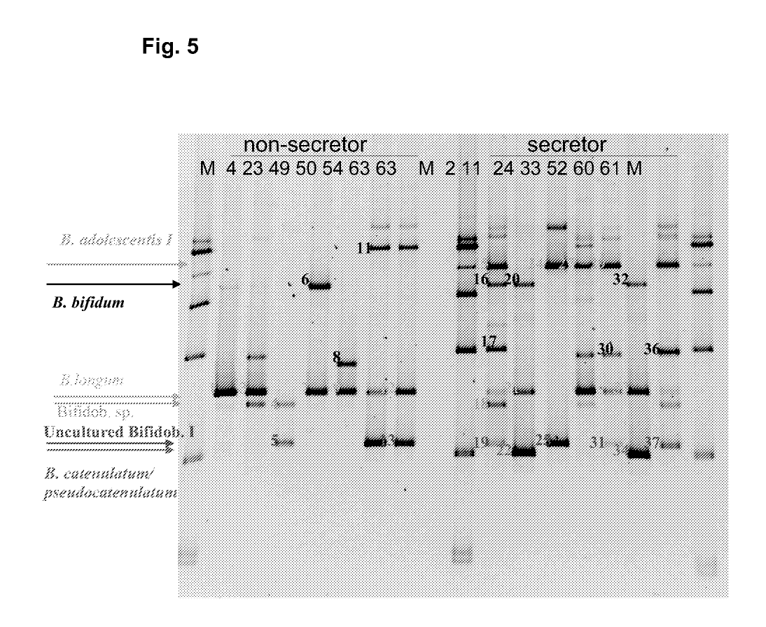

DGGE analysis and identification of the bands by sequencing was performed as described above. Identification was based on the Blast search of the sequences obtained from the excised bands of the DGGE gels. The results showed that several common bifidobacterial genotypes were missing or were present rarely in non-secretor individuals as compared to those found in secretor individuals. Specifically, most commonly detected genotypes of B. adolescentis (bands 15, 24, 27, and 29 in FIG. 5) and B. catenulatum/pseudocatenulatum (bands 22 and 34 in FIG. 5) and genotypes related to uncultured Bifidobacterium (bands 5, 13, 19, 25, 31 and 37 in FIG. 5), or those spieces and/or strains of Bifidobacterium whose detailed identification at the spieces-level requires further analyses, e.g. sequencing, were not detected in non-secretors. Moreover, genotypes related to B. bifidum (bands 6, 8, 11, 16, 17, 20, 30, 32, 36 in FIG. 5) and uncultured Bifidobacterium were more rarely detected in non-secretor individuals than in secretor individuals. The most commonly detected Bifidobacterium genotypes in the entire set of study samples were also those whose occurrence differed between the non-secretor individuals and secretor individuals (bold in Table 3), except for B. longum which was equally common in both secretor individuals and non-secretor individuals. Thus, the results indicated that non-secretors lacked or carried a low number of several Bifidobacterium genotypes, which were common in secretors. The results are presented in FIG. 5 and Table 3.

### Example 7

DGGE analysis and band position analysis using the BioNumerics-software were performed as described above. The result showed that the Bifidobacterium genotypes present in the nonsecretor individuals represented Bifidobacterium genotype 4 (band position 16.3%), Bifidobacterium genotype 6 (band position 20.4%), Bifidobacterium genotype 7 (band position 22.3%), Bifidobacterium bifidum (band position 29.7%), Bifidobacterium genotype 12 (band position 43.8%), Bifidobacterium genotype 16 (band position 47.3%), Bifidobacterium genotype 17 (band position 49.5%), Bifidobacterium genotype 18 (band position 55.0%), Bifidobacterium genotype 20 (band position 62.2%) and Bifidobacterium longum (band position 53.5%). (Table 3, FIG. 6).

TABLE 3
The identification of the band positions and the detection frequency
of bands in non-secretors (NSS, n = 6) and secretors (SS, n = 42).

| Closest relative by Blast (closest | Number of | | Total number | | | |
|---|---|---|---|---|---|---|
| cultured relative, similarity in | sequenced | Band | of bands | | | |
| Blast) | bands | position | detected | % in NSS | % in SS | % in all |
| B. longum | 7 | 53.5% | 47 | 83 | 88 | 87 |
| [symbol] a | 4 | 26.6% | 36 | 0 | 75 | 67 |
| uncultured [symbol] | 6 | 62.2% | 34 | 33 | 67 | 63 |
| ( [symbol] 475/480, 98%) | | | | | | |
| Nd | | 17.7% | 18 | 0 | 38 | 33 |
| [symbol] | 2 | 63.7% | 18 | 0 | 38 | 33 |
| [symbol] | | | | | | |
| Nd | | 20.4% | 16 | 17 | 31 | 30 |
| [symbol] | 4 | 29.7% | 16 | 17 | 31 | 30 |
| B. adolescentis b | 1 | 22.3% | 10 | 17 | 19 | 19 |
| Bifidobacterium sp | | 47.3% | 9 | 17 | 17 | 17 |
| (B. catenulatum, 477/479, 99%) | | | | | | |
| uncultured Bifidobacterium | | 43.8% | 8 | 17 | 15 | 15 |
| (B. adolescentis 476/481, 98%, | | | | | | |
| /B. ruminantium 455/457, 99%)* | | | | | | |
| uncultured Bifidobacterium | | 55.0% | 7 | 17 | 13 | 13 |
| (B. adolescentis 465/468, 99%) | | | | | | |
| uncultured [symbol] | | 44.5% | 6 | 0 | 13 | 11 |
| ( [symbol] 454/456, 99%) | | | | | | |
| Nd | | 16.3% | 5 | 17 | 8 | 9 |
| Nd | | 46.1% | 5 | 0 | 10 | 9 |
| Other (11 band positions) | | | 1 . . . 4 | 0-17% | 2-8% | 2-7% |

The band positions, which frequency differed between non-secretors and secretors are in bold.
(nd = not determined)
*The sequences of two bands classified in to this band position (43.8%) were not identical and they had similarity 97.3%

1. A probiotic composition characterized in that it is tailored based on the bifidobacterial composition of the intestine of at least one individual with non-secretor blood group phenotype.

2. The probiotic composition according to claim 1, comprising at least one strain selected from the group consisting of Bifidobacterium genotype 4, Bifidobacterium genotype 6, Bifidobacterium genotype 7, Bifidobacterium bifidum, Bifidobacterium genotype 12, Bifidobacterium genotype 16, Bifidobacterium genotype 17, Bifidobacterium genotype 18, Bifidobacterium genotype 20, and B. lognum.

3. The probiotic composition according to claim 1, further comprising at least one prebiotic agent.

4. The probiotic composition according to claim 3, wherein the prebiotic comprises fucose-containing glycans.

5. The probiotic composition according to claim 3, wherein the prebiotic is lacto-N-biose I.

6. A method of tailoring a probiotic composition based on the spectrum of bifidobacteria found from the intestine of at least one individual with non-secretor blood group phenotype.

7. Use of the secretor/non-secretor blood group status of an individual in assessing the need for bifidobacteria-enriched probiotic supplementation.

8. A method of assessing the need of an individual for bifidobacteria-enriched probiotic supplementation comprising determining the secretor/non-secretor blood group status of the individual.

9. A method of assessing the need of a breast-fed baby for bifidobacteria-enriched probiotic supplementation comprising determining the secretor/non-secretor blood group status of the baby and that of the baby's mother.

10. A use of the secretor/non-secretor blood group status of an individual in estimating a dose of bifidobacteria supplementation needed for a desired effect.

11. A method of identifying an individual at risk for suffering from a gastrointestinal disorder comprising determining the secretor/non-secretor blood group status of the individual.

12. The probiotic composition according to claim 1, wherein the composition is tailored for infants of the non-secretor type.

13. The probiotic composition according to claim 1, wherein the composition is tailored for infants regardless of their secretor phenotype, whose breast-feeding mother is of the non-secretor blood group type.

14. The probiotic composition according to claim 1, wherein the composition is tailored to elderly individuals for supporting the maintenance of bifidobacteria diversity and abundance.

15. A method for treating disorders or diseases related to unbalanced mucosal microbiota in an individual comprising administering to the individual a therapeutically effective amount of the composition of claim 1.

16. A method for treating disorders or diseases having FUT2 gene as a susceptible factor in an individual comprising administering to the individual a therapeutically effective amount of the composition of claim 1.

17. A method for treating inflammatory bowel disease, urogenital infection and/or low levels of vitamin B12 in an individual comprising administering to the individual a therapeutically effective amount of the composition of claim 1.
